SEQUENCE LISTING (31 sequences)

SEQ ID NO: 1 - protein, 50 aa, synthetic construct
QQEKNEKDLLALDSWANLWNWFDITNWLWYIKIFIMIVGGLICLRIIFAV

SEQ ID NO: 2 - protein, 7 aa, synthetic construct
SARGDLA

SEQ ID NO: 3 - protein, 40 aa, synthetic construct
TNASSEVAVLYQDVNCTDVSTAIHADQLTPAWRIYSTGNN

SEQ ID NO: 4 - protein, 39 aa, synthetic construct
TNTSNQVAVLYQDVNCTEVPVAIHADQLTPTWRVYSTGS

SEQ ID NO: 5 - protein, 12 aa, synthetic construct
GGSGGSGGSGGS

SEQ ID NO: 6 - protein, 5 aa, synthetic construct
GGSGG

SEQ ID NO: 7 - protein, 15 aa, synthetic construct
HGHGWQIPVWPWGQG

SEQ ID NO: 8 - skipped

SEQ ID NO: 9 - DNA, 12 bp, synthetic construct
  source 1..12: mol_type = other DNA
cagctattacgg

SEQ ID NO: 10 - DNA, 18 bp, synthetic construct
  source 1..18: mol_type = other DNA
atcattgattcctacttt

SEQ ID NO: 11 - DNA, 94 bp, synthetic construct
  source 1..94: mol_type = other DNA
aatgtccaattcaagctgtgatagtcgttcctcgtgctgaattcgtcagtggttcctctcccgacagttagtaactaaggatgaaatgcatggg

SEQ ID NO: 12 - DNA, 100 bp, synthetic construct
  source 1..100: mol_type = other DNA
gtccaattcaagctgtgatagtcgttcctcgtaagcttacaggtacttcagtcctcattgcaactccatacgtttagtaactaaggatgaaatgcatggg

SEQ ID NO: 13 - DNA, 100 bp, synthetic construct
  source 1..100: mol_type = other DNA
gtccaattcaagctgtgatagtcgttcctcgtaagcttatgagccctcaccctcatccgcgacaccatcacacctagtaactaaggatgaaatgcatggg

SEQ ID NO: 14 - DNA, 100 bp, synthetic construct
  source 1..100: mol_type = other DNA
aatgtccaattcaagctgtgatagtcgttcctcgtgctagcctgaccccgctgaccaccagccatctgcgcagctagtaactaaggatgaaatgcatggg

SEQ ID NO: 15 - DNA, 100 bp, synthetic construct
  source 1..100: mol_type = other DNA
tgtccaattcaagctgtgatagtcgttcctcgtgctgtgattagcaaccatgcgggcagcagccgccgcctgtagtaactaagcttgatgaaatgcatgt

SEQ ID NO: 16 - DNA, 95 bp, synthetic construct
  source 1..95: mol_type = other DNA
gtccaattcaagctgtgatagtcgttcctcgtgctggtcatcaccatcatcatcacgggtcctagtaagctagcctaaggatgaaatgcatgtgg

SEQ ID NO: 17 - DNA, 97 bp, synthetic construct
  source 1..97: mol_type = other DNA
gtccaattcaagctgtgatagtcgttcctcgtgcagcggccatcatcatcatcatcatggcagctagtaagctagcctaaggatgaaatgcatgtgg

SEQ ID NO: 18 - DNA, 104 bp, synthetic construct
  source 1..104: mol_type = other DNA
gtccaattcaagctgtgatagtcgttcctcgtgcgatgtggaatggctggatgaacgcgtgccgctggtggaaacctagtaactaagcttgatgaaatgcatgt

SEQ ID NO: 19 - DNA, 86 bp, synthetic construct
  source 1..86: mol_type = other DNA
aaatgtccaattcaagctgtgatagtcgttcctcgtgctgctagcgaagaagaagaatagtaactaaggatgaaatgcatgtctaa

SEQ ID NO: 20 - protein, 20 aa, Severe acute respiratory syndrome-related coronavirus
RYLRHGKLRPFERDISNVPF

SEQ ID NO: 21 - protein, 20 aa, Severe acute respiratory syndrome-related coronavirus
VNCTDVSTAIHADQLTPAWR

SEQ ID NO: 22 - protein, 20 aa, Severe acute respiratory syndrome-related coronavirus
GFNFSQILPDPLKPTKRSFI

SEQ ID NO: 23 - protein, 27 aa, synthetic construct
GGSGGSGGSGGSHGHGWQIPVWPWGQG

SEQ ID NO: 24 - protein, 39 aa, synthetic construct
GGSGGSGGSGGSHGHGWQIPVWPWGQGGGSGGSARGDLA

SEQ ID NO: 25 - protein, 47 aa, synthetic construct
GGSGGSGGSGGSHGHGWQIPVWPWGQGGGSGGSARGDLAGSHHHHHH

SEQ ID NO: 26 - protein, 26 aa, synthetic construct
GGSGGSGGSGGSDVEWLDERVPLVET

SEQ ID NO: 27 - protein, 38 aa, synthetic construct
GGSGGSGGSGGSDVEWLDERVPLVETGGSGGSARGDLA

SEQ ID NO: 28 - protein, 23 aa, synthetic construct
GSGRYLRHGKLRPFERDISNVPF

SEQ ID NO: 29 - protein, 23 aa, synthetic construct
GSGVNCTDVSTAIHADQLTPAWR

SEQ ID NO: 30 - protein, 23 aa, synthetic construct
GSGGFNFSQILPDPLKPTKRSFI

SEQ ID NO: 31 - protein, 22 aa, synthetic construct
GSGGFNFSQILPDPLKPTKRSF

(19) United States
(12) Patent Application Publication
(10) Pub. No.: US 2025/0368982 A1
(43) Pub. Date: Dec. 4, 2025

(54) RNA-Coliphage Q-Beta Biosensors

(71) Applicant: The Trustees of Indiana University, Bloomington, IN (US)

(72) Inventor: Alain Bopda Waffo, Carmel, IN (US)

(21) Appl. No.: 19/222,906

(22) Filed: May 29, 2025

Related U.S. Application Data
(60) Provisional application No. 63/652,757, filed on May 29, 2024.

(51) Int. Cl. C12N 15/10; G01N 33/569

(52) U.S. Cl. CPC C12N 15/1037; G01N 33/56983; G01N 2333/165; G01N 2469/20

(57) ABSTRACT

A biosensor tool that uses a novel phage Qβ display technology to monitor the presence of the biological or chemical threat. The biosensor including a probe specific to the biological or chemical threat and a detectable molecule expressed on the surface of the phage Qβ and an analyte capable of binding to the probe and blocking the detectable molecule.

### STATEMENT OF GOVERNMENTAL RIGHTS

This invention was made with government support under AG075132 awarded by National Institutes of Health, and under 2206945 awarded by the National Science Foundation. The Government has certain rights in the invention.

### FIELD OF THE INVENTION

The present disclosure relates generally to biosensors and methods of detecting the presence of organic and nonorganic molecules.

### REFERENCE TO SEQUENCE LISTING

The instant application contains a Sequence Listing XML which has been submitted electronically and is hereby incorporated by reference in its entirety. Said Sequence Listing XML copy, created on May 22, 2024 is named “IU20230440101ST26.xml” and is 28,207 bytes in size.

### BACKGROUND

The most deleterious uncertainty and problem of an imminent biological or chemical threat is detection and monitoring. For example, several RNA viruses, such as human immunodeficiency virus (HIV), yellow fever virus (YVF), Zika virus (ZIKV), Ebola virus (EBOV), SARS-associated coronavirus (SARS-COV), lassa mammarenavirus (LASV), and COVID-19 coronavirus (SARS-COV-2), are associated with significant morbidity and mortality due to their high rate of transmission. For these viral threats, the glycoprotein, envelope, and spike proteins are the major biomarkers relied upon for these infectious agents. These are the proteins that project from the surface of the virus and facilitate the viruses' entry into host cells. They are also the proteins that induce the production of neutralizing antibodies in the host.

Currently available methods for detecting chemical and biological threat agents, such as these viruses, require a combination of sophisticated biochemical and biophysical tools—each with substantial drawbacks. For example, detecting a new virus outbreak requires sophisticated and costly analytical techniques that combine serology testing, polymerase chain reaction, protein mass spectrometry, and genome sequencing. Some deleterious biological and chemical agents act at a very small concentration, making them difficult to detect. Additionally, these agents may disappear in the host after action, making them very difficult to monitor.

Aspects of the invention disclosed herein address the need for improved detection and monitoring of biological and chemical threats.

### SUMMARY OF THE INVENTION

A first aspect of the invention includes methods for creating an RNA coliphage Qβ biosensor.

A second aspect of the invention includes RNA coliphage Qβ biosensors capable of detecting the presence of a chemical.

A third aspect of the invention includes RNA coliphage Qβ biosensor capable of detecting the presence of a virus or bacteria.

A first embodiment is an RNA-colliphage Qβ biosensor for detection of an agent comprising a probe, a transducer and an analyte, wherein the probe is amino acid sequence unique to the agent; the transducer is a detectable molecule; the analyte is a molecule that binds to the probe; and wherein the transducer is positioned at the N-terminus or C-terminus of the probe; and further wherein the probe and transducer are expressed on the surface of the RNA-colliphage Qβ at the position of the RNA-colliphage Qβ A₁ protein.

A second embodiment is the RNA-colliphage Qβ biosensor wherein the agent is a biological agent.

A third embodiment is the RNA-colliphage Qβ biosensor wherein the biological agent is a virus.

A fourth embodiment is the RNA-colliphage Qβ biosensor wherein the virus is HIV, FMDV, SARS-COV, SARS-COV-2, EBOV, or LASV.

A fifth embodiment is the RNA-colliphage Qβ biosensor wherein the probe is a sequence for an epitope of a surface protein of the agent.

A sixth embodiment is the RNA-colliphage Qβ biosensor wherein the transducer is a peptide.

A seventh embodiment is the RNA-colliphage Qβ biosensor wherein the peptide is selected from the group consisting of furan, Strep II tag, Sortase A, Cytein-maleine, and Biot tag.

An eighth embodiment is the RNA-colliphage Qβ biosensor wherein the analyte is an IgG specific to the probe.

A ninth embodiment is the method of detecting SARS-COV using the biosensor comprising the steps of xxposing the biosensor to a sample containing an analyte specific to the agent for detection to, allowing the analyte to bind to the biosensor probe; detecting the presence of biosensor transducer; and comparing the detection of biosensor transducer present after exposure to the sample with a control detection of biosensor before exposure to the sample.

A tenth embodiment is the method wherein the RNA-colliphage Qβ biosensor comprises the probe SEQ ID NO:3 the transducer is Sortase A, and the transducer is positioned at the N-terminal of the probe; and wherein the analyte is an anti-S antibody.

An eleventh embodiment is the method wherein the RNA-colliphage Qβ biosensor comprises the probe is SEQ ID NO:3 the transducer is cysteine-maleine, and the transducer is positioned at the N-terminal of the probe; and wherein the analyte is an anti-S antibody.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A-1C. Schematic representation of a phage Qβ (FIG. 1A), a phage Qβ displaying a probe and an transducer peptides joined by a linker all fused to the end of the A₁ protein (FIG. 1B), and a phage Qβ biosensor comprising the Qβ displaying a probe and an transducer peptides joined by a linker all fused to the end of the A₁ protein complexed with a target (here an IgG) thereby masking the transducer portion of the A₁ protein (FIG. 1C).

FIG. 2. Schematic representation of RNA display library methodology: The recombinant cDNA of Qβ genome or other variants RT-PCR (Panel A); from any insert generated by (PCR) or a library constructed (Panel B); the vector constructed from purified Qβ cDNA in pBR322 (Panel C); the vector for display system obtained by cloning (Panel D); the generation of hybrid phages or variants after E. coli HB101 transformation (Panel E).

FIG. 3. Schematic representation of the nano-tag oligonucleotide library sequences.

FIG. 4. Schematic representation of the general organization of the recombinant Qβ with various tags, including QβA1-Biot phages with the biotin tag, QβA1BiotFMDV phages with FMDV epitopes (SARGDLA) separated by the biotin, QβBiotFMDV6H which are the same as the QβA1BiotFMDV with 6xHis-tags, QβA1Step phages with strep II tag, and QβA1StrepFMDV which are the same as the QβA1BiotFMDV with a Strep tag. A GGS linker (GGSGGSGGSGGS) was added before the nano tag and a shorter GGS linker (GGSGG) was added between the nano tag and the FMDV epitope.

FIG. 5. Image of agarose gel electrophoresis of the unpurified PCR products of the nanotag genes fused with A₁ minor coat protein gene. FIG. 5A represents the fragments of the A₁ (600 bp starting Bpu 10I) fused with nanotag gene like A₁ deleted (−150 bp) with 6xHis-tag; Strep II tag (Strep); biotin tag (Biot); avidin tag (Avi); ZnS tag (ZnS); gold tag (Au1, Au2). FIG. 5B is the same as those shown in FIG. 5A with A₁ (250bp starting Aƒl II) fused with nanotags.

FIG. 6. Images of agarose gel electrophoresis of the RNA display system vector construction analysis. FIG. 6A (top left) shows product of the recombinant pQβ7 restriction digestion lane 1-3 and 12pQβStrp with Pst I; lane 4 pQβWT digested with Pst I; lane 5-6 pQβAd2 digested Nhe I; lane 7-8 pQβBiot digested Nhe I; lane 9-10 pQβAvi digested EcoR V. FIG. 6B (bottom left) shows lanes 2-5 pQβZns, pQβAu, pQβCo, pQβCds digested NheI respectively; lane 6 pQβ7. FIG. 6C (top right) shows the RT-PCR of RNA extracted from purified phages Qβstrep, QβBiot, QβAu, QβCds, QβCo, QβZnS, QβAvi, QβHis, Qβ7, QβAd2His, QβAd2, QβAd2VG4, respectively, being fragment amplified between Afl II and Nsi I. FIG. 6D (bottom right) shows the RT-PCR of RNA extracted from purified phages of FIG. 4C being fragment amplified between Bpu 10I and Nsi I. M1 and M2 ladders are 100 bp and 10 kbp respectively.

FIG. 7. Images of dot blotting analysis of the recombinant purified QβBiot, QβStrep, and QβHis phages. QβWT being the wild type as control and D1, D2 and D3 are the 10¹⁰, 10⁹, and 10⁸ titrations of phages spotted and probed with anti-His-HRP, anti-Strep-HRP, and anti-Blot-HRP directly and respectively. All phages were dialyzed against phage buffer.

FIG. 8. Cryo-electron microscopy image of recombinant phages detected with 10 nm nickeled gold (NTA), with FIG. 6A being Qβ7 wild type control; FIG. 6B being QβStrepHis; FIG. 6C being QβHis; and FIG. 6D being QβBiotHis.

FIG. 9A. Plot analysis of optical density (OD) of the horseradish peroxidase (HRP) product vs increase concentration of anti-His-HRP antibody (plate coated with QβHis).

FIG. 9B. Plot analysis of optical density (OD) of the horseradish peroxidase (HRP) product vs increase concentration of streptavidin conjugated to HRP (plate coated with QβStrep).

FIG. 9C. Plot analysis of optical density (OD) of the horseradish peroxidase (HRP) product vs increase concentration of biotin conjugated to HRP (plate coated with QβBiot).

FIG. 9D. Plot analysis of OD vs increase concentration of SD6 antibody against FMDV immunogenic epitope (plate coated with QβBiotFMDV) with a fix concentration of Biotin-HRP (10 μg/ml).

FIG. 10. Schematic representation of RNA coliphage Qβ insert of spike protein derived fragments from design to motif sequence selection. The top schematic showing the known O- and N-glycosylation functional domains of the spike protein organization from the N-terminus signal sequence (F1), middle sequence with cleavage site (S1/S2) to the C-terminus (F14); the middle schematic showing the phage Qβ genome organization of cDNA with non-coding region (NCR), maturation protein (A2/MA2), coat protein (Cp), read-through protein (A1/MCPA1) with the insertion cassette at the end (blue) for cloning, the replicase protein (Rep); the bottom schematic showing the residue motif of different epitopes (EP) obtained within the cassette on the Qβ phage; the phage with epitope 1 (QβEP1), the phage with epitope 2 (QβEP2), the phage with epitope 3 (QβEP3), the phage with epitope 3 with a deletion mutant of I (Qβ [symbol] EP3).

FIG. 11A-D. Agarose gel image analysis of the results of SARS-COV S protein gene fragments. (A) Amplification: PCR products of 14 overlapping fragments amplification fusion PCR of a portion of A1 and of the S gene from F1 to F14 (˜800 bp); M2 and M1 are 10 kb and 100 bp ladder respectively. WT is the A1 gene amplified and Ad2 is the delete A1 gene amplified as control without the C-terminus 150 bp gene portion. (B and D): Construction of recombinant phage vector for RNA phage display S fragments. Positive recombinant pQβF1, pQβF4, pQβF6, pQβF8, pQβF11, and pQβF13 plasmids are linearized with Not I respectively; positive recombinant pQβF2 and pQβF12 were digested into 700 bp and 7 kbp fragments with EcoRV respectively; positive recombinant pQβF3 and pQβF9 were digested into 1, 2.3, and 4.4 kbp with Nde I respectively; positive recombinant pQβF5 and pQβF10 were digested with Nhe I into 1 and 6.7 kbp fragments respectively; positive recombinant pQβF7 and pQβF14 were digested with Pst I into 3 and 4.7 kbp fragments respectively; M1 and M2 are 1 kbp and 100 bp DNA ladders respectively. The plasmid pQβAd2 is a negative control wildtype with A1 deletion linearized with Not I. (C) The RNA genotype size analysis for recombinant phages: From GF1 to GF14 are the genomic portion of the recombinant phages with S fragment 1 to 14 respectively, amplified by RT-PCR. GWT and GAd2 are the same genomic portion on the wild type and A1 deletion phages respectively, amplified by RT-PCR as negative controls. M1 and M2 are 1 kbp and 100 bp DNA ladders, respectively.

FIG. 12A-B. SARS-COV S protein epitopes reactivity analysis. (A): Dot blotting analysis of the recombinant purified phages harboring the epitope (EP) EP1 (F6: S425-460); EP2 (F7/F8: S601-620); EP3 (F10: S781-800); and EPCh (chimeric of EP1, EP2, and EP3). WT is the control wild type; RBM1 is the hACE2 receptor binding motif of SARS-COV. The concentration of recombinant phages was 1012 pfu/ml, respectively. (B): ELISA Diagram analysis of S protein selected epitopes (EP). EP1 (F6: S425-460); EP2 (F7/F8: S601-620); EP3 (F10: S781-800); and EPCh (chimeric of EP1, EP2, and EP3). WT is the control wildtype. The concentration of recombinant phages was 105 pfu/ml, respectively.

FIG. 13A-B. SARS-COV S protein chimeric epitope reactivity analysis. (A): Dot blotting analysis of the recombinant purified phage harboring the chimeric epitope EPCh (chimeric of EP1, EP2, and EP3). WT is the control wild type; from PFChD4 to PFChD1 are 108, 109, 1010, and 1011 pfu/ml, respectively. (B): ELISA with recombinant phages QF6. The RBS1-ACE2 interaction: the plot analysis of optical density (OD) of the horseradish peroxidase (HRP) product of anti-rhACE2 vs. increase of concentration of hACE2.

FIG. 14. Schematic representation of the general organization of the recombinant Qβ phage with the chimeric epitope insert and tags: Qβ phage genome organization with the maturation protein (A2), the coat protein, the read-through protein (A1) with the insertion cassette (S-FT) at the end for cloning, and the replicase protein (Repd). The cloning cassette contains the chimeric epitope (Epitope3-Epitope1-Epitope2) and the different biosensing tags LPETG, Biotin Tag (BIOT-TAG), and Streptavidin tag (STREP-TAG) at the C and N-termini of the chimeric epitope.

### DETAILED DESCRIPTION

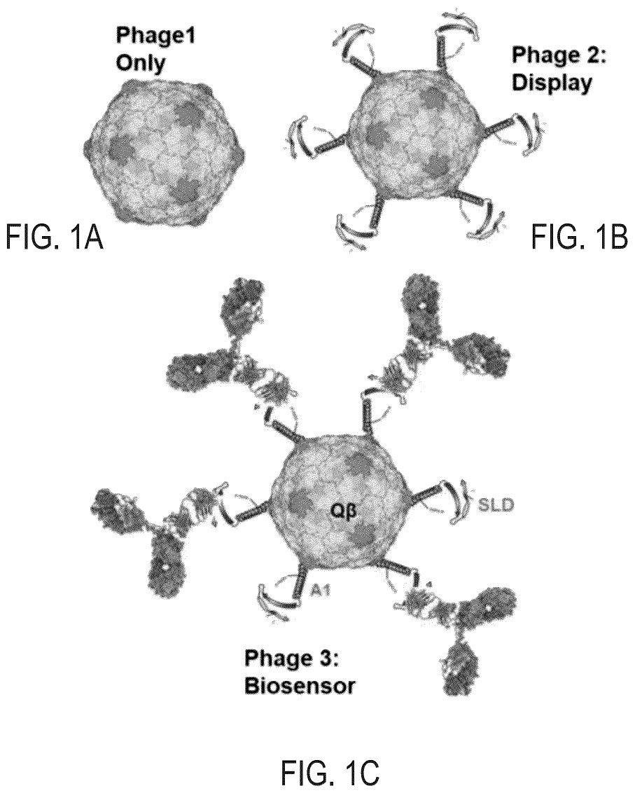

Described herein is a biosensor tool that uses a novel display technology to monitor the presence of the biological or chemical threat. The biosensor tool utilizes the phage Qβ. The phage Qβ is a small, positive stranded RNA virus, a member of Leviviridae that infects bacteria with the F⁺ pilus, such as E. coli. (FIG. 1A). Previously, phage engineering was mostly applied to DNA phages, but DNA phages prevent any rapid evolution or adaption. RNA phages possess features that can fuel and accelerate in vitro evolution and adaption. Specifically, the RNA-dependent-RNA-polymerase (RdRp) of phage Qβ does not have proofreading activity, resulting in higher mutation rates. The high mutation rate of the RdRp simulates in vitro evolution and affinity-maturation important for developing probes against a biological or chemical target with bio-panning.

The infectious Qβ virion includes only four genes in a genome size of 4220 nucleotides. The genes encode the subunit II (β) a replicase, a major coat protein, a maturation protein (referred to as A2 or A₂) and a read-through minor coat protein (referred to as A1 or A₁). The minor coat protein A1 shares the same initial codon with the coat protein and is produced during the translation when the coat protein stop codon UGA triplet (about 400 nucleotides from initiation) is suppressed by low level of ribosome mis-incorporation of tryptophan at the coat protein termination signal. As described herein, the surface of phage Qβ was engineered through the A1 protein to present a library of peptides without affecting the infectivity of the phage.

Phage Qβ can present 12 copies of foreign appropriate affinity peptide(s) on its exterior surface in uniform distribution at the corners of the phage icosahedron. (FIG. 1B).

Prior phage display libraries and biosensor development technologies were exclusively designed and executed with filamentous DNA phage M13 (Goldman, E. R., et al., J. Mol. Recognit., 13(6), 382-387; Lee, J. M., et al., Biosens. Bioelectron., 188, 113339). The M13 minor coat proteins (pIII) are located only at one end of the filamentous phage and are not equally distributed upon its surface like the newly developed icosahedral Qβ RNA phage. Additionally, due to this structural restriction, the prior M13 replication system is less adapted to evolutionary modifications relative to Qβ.

The biosensor described herein utilizes a complex comprising three primary components: (1) probe, (2) analyte, and (3) transducer. (FIG. 1C).

The probe is an amino acid sequence displayed on the exterior of the phage that is unique to the agent to be detected. The probe may be unique to a specific epitope of a biological agent, such as a virus or bacterial threat. Exemplary probes include amino acid sequences to epitopes of the glycoprotein gp41 of HIV, spike protein of SARS-COV, spike protein of SARS-CoV-2, glycoprotein of EBOV, or glycoprotein of LASV.

The analyte is a molecule capable of binding to the probe of the biosensor. The analyte may be an IgG specific to the epitope of the biological agent that is produced after infection by or vaccination against the targeted biological agent.

The transducer is a detectable molecule. The transducer may be a peptide, chemical, nanobody, and/or nanotag for detection. We have mapped strong material binding peptides that can be used as transducer. Charged amino acids can lose their charge and change the potential of the solution may also be used as transducers. Exemplary transducers include but are not limited to furan, StrepII tags, Sortase A, Cyteine-maleine, or Biot tags. Those of skill in the art will appreciate that the environment of the analyte (such as plasma, blood, urine, surface swab, etc) may inform the selection of the transducer.

As described herein, the positioning of the probe with respect to the transducer is key to obtaining a probe-to-analyte signal by the biosensor. Several exemplary embodiments of the present biosensor for detection of RNA viruses are set out in Table 1 below:

| Viral | Probe | Transducer | Analyte | Positioning |
|---|---|---|---|---|
| Threat | (P) | (T) | (A) | of P |
| HIV | Glycoprotein | Furan | Ani-gp41 | N-terminus |
| | gp41 | | antibody | |
| | (SEQ ID NO: 1) | | | |
| FMDV | (SEQ ID NO: 2) | StrepII tag | SD6 | C-terminus |
| | | | antibody | |
| SARS- | Spike protein | Sortase A | Anti-S | N-terminus |
| CoV | (SEQ ID NO: 3) | | antibody | |
| SARS- | Spike protein | Cyteine- | Anti-S | N-terminus |
| CoV-2 | (SEQ ID NO: 4) | maleine | antibody | |
| EBOV | Glycoprotein | Biot tag | Anti-GP | C-terminus |
| | | | antibody | |
| LASV | Glycoprotein | Strep II tag | Anti-GP | C-terminus |
| | | or Biot tag | antibody | |

The SEQ ID NOs referenced for the exemplary probes in Table 1 correspond to the following amino acid sequences:

| SEQ ID | |
|---|---|
| NO: | AA Sequence |
| 1 | QQEKNEKDLLALDSWANLWNWFDITNWLWYIKIFIMIVGG |
| | LICLRIIFAV |
| 2 | SARGDLA |
| 3 | TNASSEVAVLYQDVNCTDVSTAIHADQLTPAWRIYSTGNN |
| 4 | TNTSNQVAVLYQDVNCTEVPVAIHADQLTPTWRVYSTGS |

As shown in FIG. 1C, the biosensor having the modified A1 proteins containing the probe and transducer is exposed to a composition potentially containing the analyte. The analyte will bind to the probe, thereby blocking the transducer. Those transducers not blocked by the analyte: probe binding are accessible to provide a signal for detection. Without analyte present, the detection signal is 100% which provides the detection reference standard. The detection signal with analyte present is then subtracted from the reference standard to quantify the target analyte detected by the biosensor.

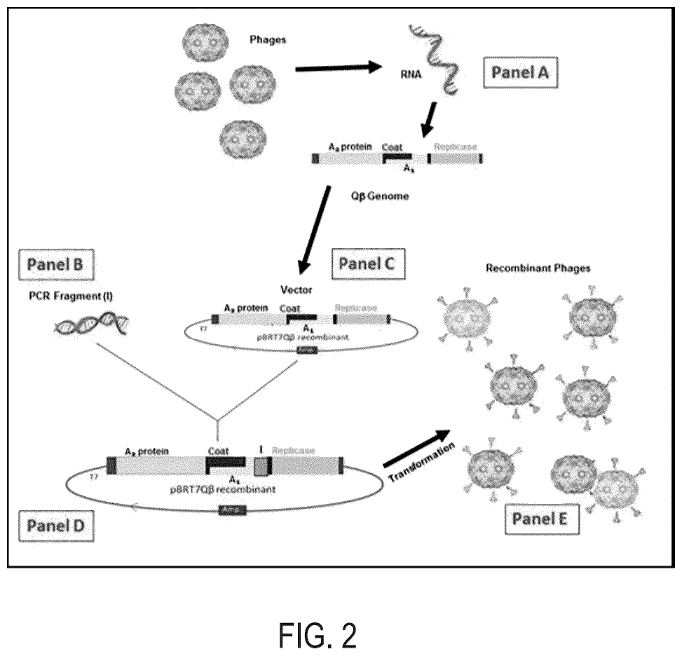

As shown in FIG. 2, the method of creating the RNA phage Qβ biosensor includes: creating a recombinant cDNA wherein a target-specific sequence (PCR fragment) is inserted within the A1 gene of the Qβ genome (FIG. 2, Panels A-C); then a vector is constructed comprising the recombinant cDNA (FIG. 2, Panel D); transforming E. coli with the vector to generate a library of hybrid Qβ phages or variants (FIG. 2, Panel E); and selecting the those hybrid phages or variants for use as a biosensor. As described herein, the phage Qβ display library of peptides can be used to bind and concentrate a broad range of organic (including but not limited to virus glycoproteins, virus-related antibodies, toxins of harmful algal blooms and bacteria) or nonorganic molecules (including but not limited to gold, zinc, cobalt, or peroxide as a precursor for explosives like TATP).

Selection of the hybrid phages capable of identifying and binding the selected target may be performed using biopanning. The term “biopanning” refers to an affinity selection technique which selects for peptides that bind to a given target. Biopanning may include conjugating the hybrid phage library to the desired target, washing away unbound phages, and eluting the bound phages. The process of conjugating, washing and eluting may be repeated until the hybrid phages with highest specificity are identified. Once the desired level of specificity is achieved, the hybrid phages may be isolated to be utilized as a biosensor. Prior to storage, the isolated phages are amplified, scaled and sequenced. The Qβ biosensor is easily scaled and is resistant to extreme conditions.

The sequences inserted into the Qβ A1 gene may be 15nt, 18nt, 21nt, 24nt, 27nt, 30nt, 33nt, 36nt, 39nt, 42nt, 45nt, 48nt, 51nt, 54nt, 57nt, 60nt, 63nt, 66nt, 69nt, 72nt, 75nt, 78nt, 81nt, 84nt, 87nt, 90nt, 93nt, 96nt, 99nt, 102nt, 105nt, 108nt, 111nt, 114nt, 117nt, 120nt, 123nt, 126nt, 129nt, 132nt, 135nt, 138nt, 141nt, 144nt, 147nt, 150nt, 153nt, 156nt, 159nt, 162nt, 165nt, 168nt, 171nt, 174nt, 177nt, 180nt, 183nt, 186nt, 189nt, 192nt, 195nt, 198nt, 201nt, 204nt, 207nt, 210nt, 213nt, 216nt, 219nt, 222nt, 225nt, 228nt, 231nt, 234nt, 237nt, or 240nt in length.

The sequence inserted within the A1 gene may optionally include a linker to maintain the correct reading frame of the recombinant protein. Linkers may include GGS linkers such as a longer GGSGGSGGSGGS linker (SEQ ID NO: 5) or a shorter GGSGG linker (SEQ ID NO: 6).

Biosensors of the present invention may be created to detect a variety of targets of interest—both nonorganic targets and organic targets. Organic targets include, but are not limited to, virus glycoproteins, virus-related antibodies, toxins of harmful algal blooms and bacteria. Nonorganic targets include, but are not limited to, gold, zinc, cobalt, or peroxide precursors of explosives like TATP. In embodiments where the target is an organic target, the inserted sequence may be selected based upon sequences (epitopes) of known proteins present on the surface of the target organism.

In some embodiments of the present invention the Qβ biosensor is adapted to detect a domain of the spike protein of the SARS-COV-1 and SARS-COV-2 viruses. While it is presently known that the spike protein produces the antibody response in humans, the specific portion of the spike protein required to produce the response is not known so the entire spike protein is currently being utilized in current vaccination and detection of SARS-COV strains. The methods described herein allows the key residues to be identified and utilized for screening by using the patient antibodies in the creation of the biosensor. In these embodiments, the functional domain(s) of the spike protein can be fused to a transducer peptide via a linker, which can then be presented on the exterior surface of the RNA Qβbiosensor. This provides a method to concentrate, detect, titer and monitor specific SARS antibodies in real time.

In some embodiments of the present invention the Qβ biosensor is adapted to detect the presence of Leviviridae (Fiersviridae) virus, which infects bacteria with the F+ pilus, is found in wastewater and is resistant to extreme conditions. As Qβ is very stable at various temperatures, we have successfully produced large scale with high titer.

In some embodiments of the present invention the Qβ biosensor is specific to nonorganic molecules. In these embodiments, the hybrid Qβ library can be used to map peptides binding to a chemical (explosives or precursors).

E. coli hosts that may be utilized in the present methods for the production of the Phage Qβ include HB101, Q13, K12 or Hfrh.

Biosensors described herein may be used to detect a biological or non-biological agent by first determining a control detection level of the transducer of the biosensor prior to exposure to a sample potentially containing the agent for detection. Then exposing the biosensor to a sample containing an analyte specific to the agent for detection. After a sufficient time for the analyte to bind to the biosensor probe, detecting the presence of biosensor transducer in the sample. This will detect the amount of transducer that is not blocked by the binding of the analyte to the probe. Comparing the detection of biosensor transducer present after exposure to the sample with a control detection of biosensor before exposure to the sample, to determine the amount of agent present in the sample.

### EXAMPLES

### Example 1: RNA Qβ Phage Displaying 15-mer Library

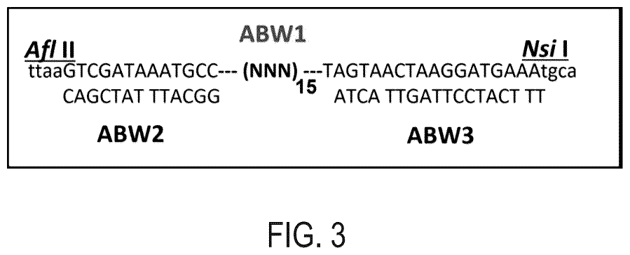

Previously, we successfully inserted and displayed a 5-mer library into the A1 minor coat protein of recombinant Qβ stably. The FMDV epitope was selected, enriched, and amplified from the 5-mer library revealing a non-canonical epitope (Skamel, C. et al., PloS One, 9(11), e113069). Herein, we extended the library size to a 15-mer within the truncated A1 for broad selection and nanotag development. The design and principle of phage display library insertion on Qβ cDNA are depicted in FIG. 2. The first challenge was to obtain the library of RNA phages with at least 109 plaque forming units/ml (pfu/ml) variants. Pooling phages from small ligation (10 ng of insertion) with a single round of amplification with E. coli K12 gave the appropriate titer for use in biopanning. The library was successfully fused at the end of the A1 minor coat protein gene sequence terminating in two natural opal and ochre stop codons, TGA and TAA respectively. The restriction enzyme sequences flanking the library (ABW1, SEQ ID NO:1, FIG. 3), were used to insert the randomized sequence at the end of the A1 gene. ABW1 is a randomized sequence synthesis for library generation and population of variant phages production against proteins and materials selection. Using the restriction enzyme sites Afl II (position 2159) and Nsi I (position 2350), 192 nucleotides were deleted at the end of A₁. The ABW2 sequence is complementary to the N-terminus of the library ABW1 flanked AFl II restriction enzyme sequence. The ABW3 sequence was complementary to the C-terminus of the library ABW1 flanked Nsi I restriction sequence. The deletion of A₁ and extension with the library were exploited to produce variants for this evolutionary library. Additionally, a specific Shine Dalgarno (SD) sequence (TAAGGAGG) was added to the intact intercistronic region (position 2339) thereby improving the production of phage plaques on the bacteria lawns (E. coli K12 and Q13). The resulting truncated A₁ can accommodate the library and confirm the key role of the intercistronic region between the A₁ and the replicase genes in recombinant Qβ phage production.

Several plasmids were created with the A₁ modification, both with the SD sequence pQβAd2SD and without the SD sequence pQβAd2. The plasmid pQβAd2SD produced recombinant phages with titer close to the wildtype, while the pQβAd2 expression titer was 3 folds lower. A recombinant phage library with titer up to 10⁹ pfu/ml of phages was successfully obtained which was enough for subsequent selection through our optimized panning strategy.

### Example 2: Biotin and Biotinylated RNA/Peptide Binding Sequences

The goal of this study was to develop biotin-binding peptides using an RNA phage display system and test whether the same peptides could be used as probes to detect and quantify biotin or biotinylated entities. Captured biotin and biotinylated entities were separately immobilized on a plate and the ORF phage library were enriched by binding to the reaction platform in the same manner as described above. The recombinant phage particle with the A₁ probe extended by the library can be anchored through the interaction between the specific probe displayed and the biotin bound target. When anchored on its target after several washes, the recombinant phage is amplified through the A2 by adding a fresh log phase (OD₆₀₀=0.7) E. coli K12 culture. The recombinant phages are then eluted by infection and used to bound another biotin target. Six rounds were performed with phages obtained during each round sequenced. A 10⁹ pfu/ml population of recombinant phage was obtained with the 15-mer library inserted into the A₁ and used to selectively identify HGHGWQIPVWPWGQG (SEQ ID NO: 7) a biotin-specific binding peptide. The predominant sequence binding biotin or biotinylated entities was: HGHGWQIPVWPWGQG (SEQ ID NO: 7) with the IPVW motif present in weaker binder. We reasoned that the IPVW motif gained fitness and was selected, amplified, and enriched to the final peptide binding biotin. After many rounds of enrichment, the biotin selective recognizing peptide was compared to weak binder candidates to decipher an IPVW common motif. The tetrapeptide motif is an isolation result of six rounds of biopanning with selective pressure in the presence of biotin from a spectrum of variants generated.

### Example 3: Biosensor With Biotin-Binding Peptide

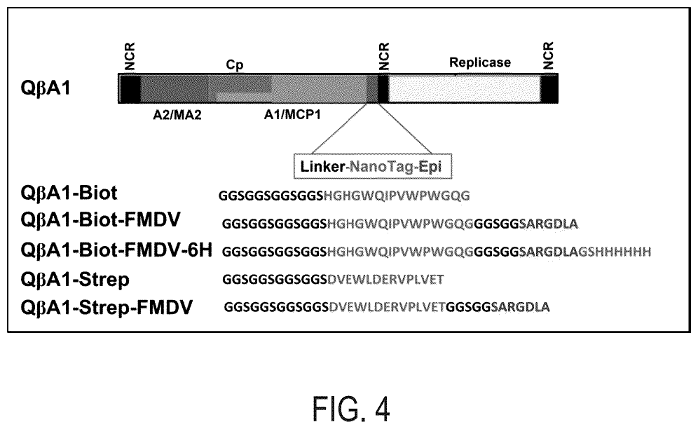

To design and generate plasmid vector variants of probe, linker, or transducer, the pQβ7 was used. pQβ7 is the vector utilized to create the phage biosensor. Several plasmids were constructed with amino acid sequences positioned as shown in our illustration in the expression cassette (FIG. 2) which were used in frame to A₁ to generate recombinant phages. As shown in FIG. 4, the QβA₁ insertions included pQβStep (streptavidin tag), pQβBiot (biotin tag), pQβBiotFMDV (biotin tag with FMDV epitope), pQβBiotFMDV6His (biotin tag with FMDV epitopes fused with His-tag), and pQβStrepFMDV (streptavidin tag with FMDV epitope). The FMDV epitope was SARGDLA. Separately, the peptide tag, linker, epitope, and His-tag were all obtained by PCR within the reverse primer while the forward primer was part of the phage cDNA sequence.

TABLE 1
List of oligonucleotide names and corresponding sequences. Bolded
sequences correspond to the phage A₁ gene portions.

| | | SEQ |
|---|---|---|
| Name | Sequence | ID NO: |
| ABW1 | ttaaGTCGATAAATGCC (NNN)15 TAGTAACTAAGGATGAAAtgca | 8 |
| ABW2 | CAGCTATTACGG | 9 |
| ABW3 | ATCATTGATTCCTACTTT | 10 |
| Au1 | AATGTCCAATTCAAGCTGTGATAGTCGTTCCTCGTGCTgaattCgtcagtggttc | 11 |
| | ctctcccgacagttagTAActaaggatgaaatgcATGgg | |
| Au2 | GTCCAATTCAAGCTGTGATAGTCGTTCCTCGTaaGCTtacaggtacttcagtcctcat | 12 |
| | tgcaactccatacgtttagTAActaaggatgaaatgcATGgg | |
| Silica | GTCCAATTCAAGCTGTGATAGTCGTTCCTCGTaaGCTtatgagccctcaccctcatc | 13 |
| | cgcgacaccatcacaccTAGTAActaaggatgaaatgcATGgg | |
| Cds | aatgtccaattcaagctgtgatagtcgttcctcgtgctAGCCTGACCCCGCTGACCA | 14 |
| | CCAGCCATCTGCGCAGCtagTAActaaggatgaaatgcATGgg | |
| ZnS | tgtccaattcaagctgtgatagtcgttcctcgtgctGTGATTAGCAACCA | 15 |
| | TGCGGGCAGCAGCCGCCGCCTGtagTAActaagCTTgatgaaatgcATGT | |
| 6xhis-tag | gtccaattcaagctgtgatagtcgttcctcgtgctGGTCATCACCATC | 16 |
| | ATCATCACGGGTCCtagtaaGCTAGCctaaggatgaaatgcatgtgg | |
| Biotin-tag | Gtccaattcaagctgtgatagtcgttcctcgtgcagcggccatcatcat | 17 |
| | catcatcatggcagcTAGTAAGCTAGCctaaggatgaaatgcatgtgg | |
| Strep II-tag | GtccaattcaagctgtgatagtcgttcctcgtgcGatgtggaatggctggat | 18 |
| | gaacgcgtgccgctggtggaaaccTAGTAActaagCTTgatgaaatgcATGT | |
| Co | aaatgtccaattcaagctgtgatagtcgttcctcgtgctGCTa | 19 |
| | gcGAAGAAGAAGAAtagTAActaaggatgaaatgcATGTCTAA | |

Modelling of A₁ with the peptide inserted at the C-terminus was conducted. The 3D structures obtained were derived from the A1 in Qβ wt, QβStrep, QβBiot, and QβAu, respectively. The N-terminus of the minor coat protein displaying peptides was not significantly changed in comparison to the wt. Structurally, the A₁ bearing additional peptide conserved its α-helixes and β-sheets in all models. For the C-terminal of A₁ with the inserted modification, only minor rotations were observed. All additional peptides to A₁ were confirmed to be exposed around the β-sheets and pointing to the outer surface of the capsid. This result was consistent to previous 3D models of A₁ fused proteins that we had performed.

A software program (such as RNA-Fold®) was used to predict the secondary structure of the RNA 3′ untranslated region of the replicase after any tag and the Shine Dalgarno sequence insertions. The region between the A₁ stop codons (2331) and the replicase start codon (2353) was checked for the availability of the start codon upon insertion of 100 to 200 nucleotides and different constrains of the newly formed hairpin with the stable tetraloop motif on this region. Any 5′ replicase domain constrain too close was optimized to fit the known secondary structure model for the two distal domains of the Qβ RNA. The plasmid pQβBiotFMDVHis was found to contain those constrains and produced a low titer of phage 10⁴ pfu/ml. The titer was brought to 107 pfu/ml after C (CAC) substitution to U (CAU). The RNA secondary structure prediction has therefore contributed to the optimization of the display system on this single-stranded RNA phage.

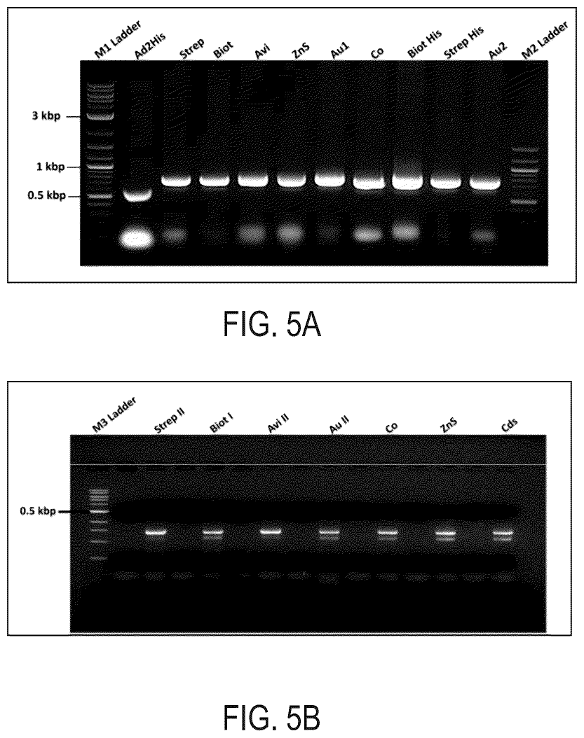

Recombinant phage vector construction strategy and genetic analysis. The reverse primers containing the gene fusion of all designated tags separately, the partial A₁, the linkers, and the natural stop codons of the phage at this region were used with the forward primer to generate the fragments shown in FIGS. 5A and 5B using pQβ7 as a template. The results suggested that the gene fusion was obtained for all tags with both different reverse primers, separately. These fragments are critically important in the construction of the recombinant plasmids for biosensor expression using the template vector. The small fragment products of PCR contained the truncated A₁ and was previously tested to produce viable phages. The large fragments were closed between Bpu 101 and Nsi I, while their small counterpart was between Afl II and Nsi I respectively. Two different constructions were made with each tag and successfully analyzed with restriction enzyme. The recombinant plasmid bearing the tag gene was recognized within the gene by the unique restriction enzyme sequence. Identifying the unique sequence of the inserted gene tag in combination with a vector sequence gave a fragment easily remarkable on the gel as presented in FIG. 5 left top and bottom. The results indicated the presence and the position of a particular sequence and its distance from another that was confirmed by Sanger sequencing with home designed forward and reverse primers to cover twice the inserted genes. The list of restriction enzymes present on the tag genes and pQβ7 vector were as follows: PstI, Nhe I, and EcoR V. The successfully sequenced recombinant plasmids were obtained in all positive restriction analysis. Additionally, the perfect expression frame was maintained for each of the recombinant plasmid vectors prior to phage expression and production.

The production of recombinant phage was done using an F⁻ E .coli HB101 avoiding premature evolutionary evens. Since the platform infects F+ bacteria for cycle and evolution, using F− bacteria prevents evolutionary changes caused by Qβ's lack of transcriptional editing. On the lawn of the indicator bacteria (k12 or Q13) after 12 hours, the plague sizes ranged from 0.4-2 mm in diameter in both wt and recombinant phages with similar titers (Table 2). The phages containing the truncated A₁ were predominantly made up of small size plaques while the rest of the wildtype and recombinant were large. These results corroborated our early report and showed that all recombinant plasmids produced plaque-like phages on the lawn of indicator bacteria. The recombinant and wt phage titers were similar between 10⁷ and 10⁹ pfu/ml (Table 2) with exception of the phages exposing the His-tag that were improved after codon optimization and secondary RNA structure revisited (from 10⁴ to 10⁷ pfu/ml). This first-generation phage titer is F⁻ bacteria was sufficient as a biosensor but for experiment purposes were further amplified and stored. Sanger sequencing, dot blotting, ELISA, and cryo-EM were used to confirm the genotype and phenotype of each recombinant phage.

TABLE 2
Comparison of recombinant phage titers from
different generations (from 1^{st} to 3^{rd})

| | phages | | | |
|---|---|---|---|---|
| Host | Qβ | QβHis | QβStrep | QβBiot |
| E. coli | 10⁹ pfu/ml | 10⁸ pfu/ml | 10⁷ pfu/ml | 10⁷ pfu/ml |
| HB101(1ₛₜ) | | | | |
| E. coli | 10¹² pfu/ml | 10¹⁰ pfu/ml | 10⁹ pfu/ml | 10⁹ pfu/ml |
| Q13 (2_{nd}) | | | | |
| E. coli | 10¹⁴ pfu/ml | 10¹² pfu/ml | 10¹¹ pfu/ml | 10¹¹ pfu/ml |
| K12 (3_{rd}) | | | | |

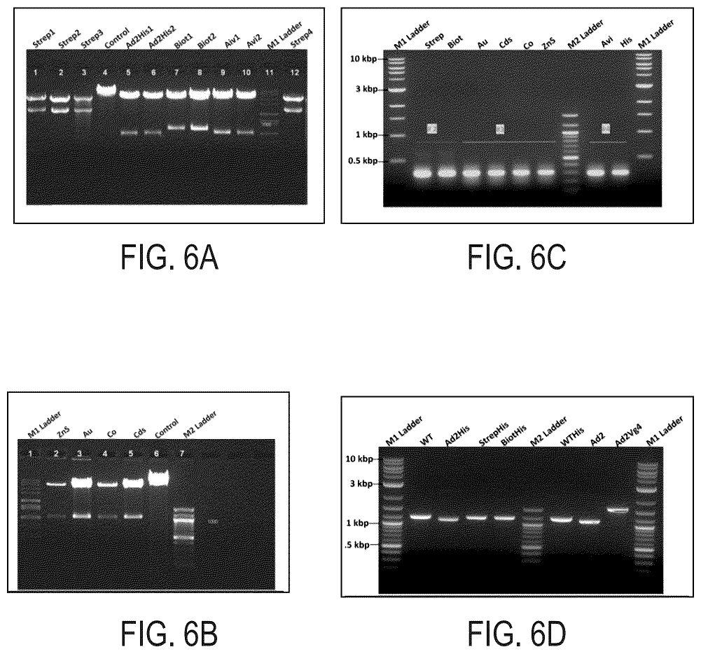

Genotype analysis of recombinant phages. After six rounds of panning the phages from single plaque were extracted, the RNA converted to cDNA, copied by RT-PCR, and sequenced. Initially, the secondary structure of native RNA was compromising the RT-PCR and an increase in intensive heat was applied to the RNA solution before the addition of the primer and reagents for reverse transcription (RT). Two sets of primers pair were used to copy the cDNA region of the genome. The amplified cDNA was successfully analysed by agarose gel electrophoresis. The result is shown in FIG. 6A-D with the appropriate band size of 250 bp (around the insert within the cloning cassette) and bottom are the band size of 1.5 kb. The large fragment was sequenced for each recombinant phage and found to contain the appropriate sequence fused in frame with the A₁ gene of the recombinant phage. Sequencing results confirmed the fragment band sizes on agarose gel and further confirmed that each recombinant phage genome was obtained.

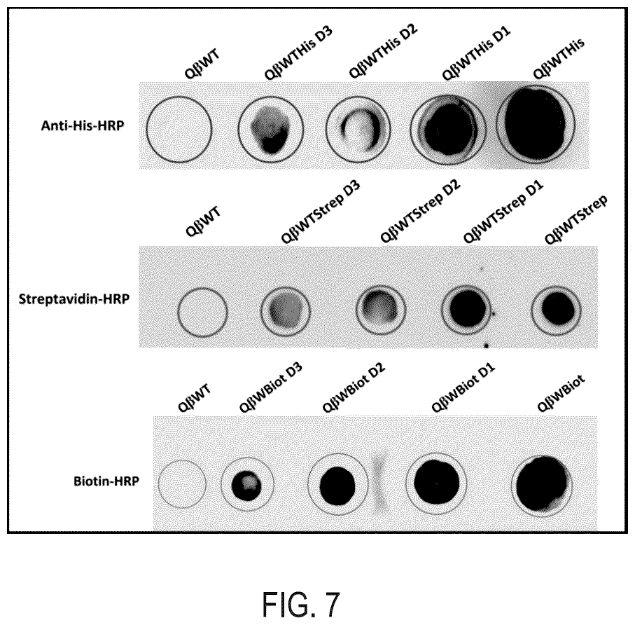

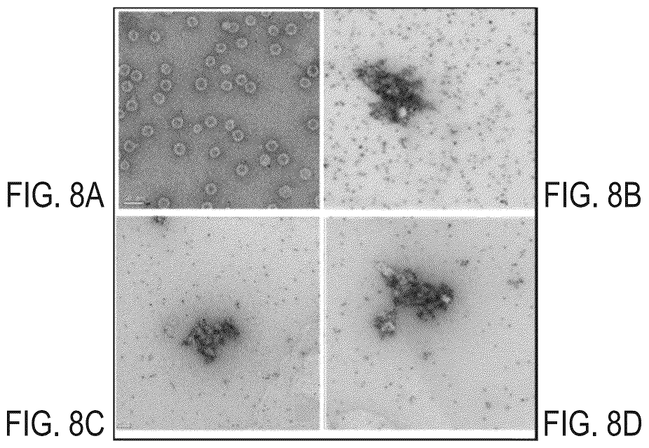

Phenotype analysis of recombinant phage. To analyse the probe (nanotag) presented on the recombinant phages, three different dilutions of each stock of phages were dialyzed and directly applied to a nitrocellulose membrane and probed with the appropriate antibody conjugated with horseradish peroxidase (HRP). The results showed a lineal presence of the nanotag on the spotted phages. The His-tag, the Strep II tag, and the newly developed biotin-tag reacted proportionately with the anti-His, streptavidin, biotin all conjugated with HRP, respectively (FIG. 7). The turnover of dark spots on the membrane reflects an increased presence of the canonical peptides (phenotype) on the recombinant phage surface recognizing and binding proportionately to the appropriate proteins. The antibody and proteins conjugated to HRP revealed the phage phenotype and was confirmed by cryo-EM and ELISA. For high visibility, recombinant phages bearing the biotin-tag and Strep II tag were separately flanked with His-tag and detected 10 nm Ni-NTA-Nanogold microscope (FIG. 8A-8D). The results showed the binding of the incorporated metal chelated to a His-tagged peptide on the phage (visible shape-like phage, less than 50 nm diameter). The extension of peptide tags detected with Ni-NTA-Nanogold was the confirmation of the phenotype of the recombinant phages.

### Competitive ELISA Between Biotin-HRP and SD6 With QβBiotFMDV Recombinant Phages

The selected biotin-binding peptide was subjected to comparative analysis with well-known peptide like Strep II tag and His-tag together with peptides binding streptavidin and anti-his tag proteins, respectively, in order to learn more about its affinity to biotin.

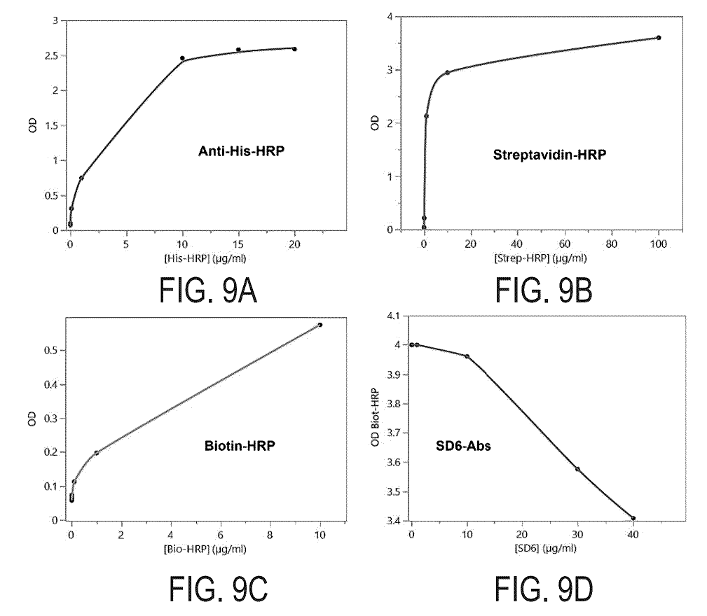

Standard ELISA was initially carried with each tag or epitope to determine the saturated concentration of the antibody or appropriate proteins. Recombinant phage titer of 10⁷ pfu/ml coated and saturated the ELISA plates. Proteins bound to the peptide tag with a hyperbolic curve, following a simple pattern of linear increase of binding to saturation (FIG. 9A-9C). Next, an ELISA was performed with an increase concentration of SD6 vs a saturated concentration of Biotin-HRP (FIG. 9D). The OD of the HRP product was recorded, plotted, and showed a decrease in HRP reactivity with increasing SD6 antibodies. Finally, our results showed that a potential binding of SD6 (anti-FMDV epitopes) preventing the biotin-HRP protein from accessing its appropriate probe tag in a competitive manner. The tags and epitopes were accessible on the surface of the phage and were close to each other in the case of the fusion to promote detection and competition.

Our data showed that the tags presented separately on Qβ phage surface, had similar affinity to their corresponding proteins. A maximum titer of 10⁷ pfu/ml of recombinant phages saturates plate bottoms by binding to 2 μg of protein signal.

### Probe: Transducer Positioning

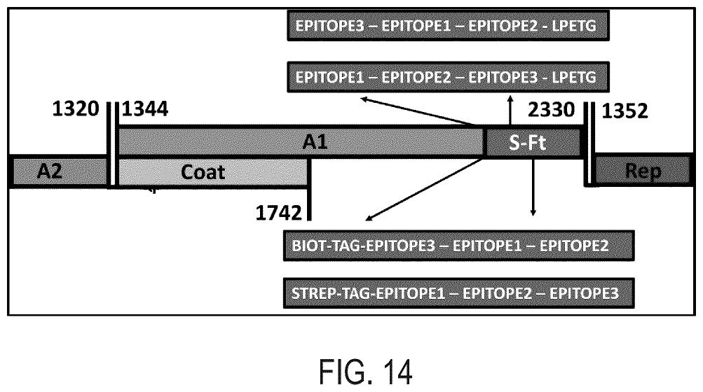

Structural conformation and the position of the components of the recombinant Qβ biosensor are important. When the FMDV probe was used with a His-tag transducer, regardless of whether a linker was used, the His-tag transducer was only accessible when presented at the extremity of other peptides or A1 (or C-terminus). Both the FMDV epitope and His-tag affinities to their cognate antibodies were abolished when fused to the N-terminus of any of the nanotags, including the Au1 tag peptide. This drastic change of the analyte SD6 in the affinity of the FMDV epitope due to its position is likely the consequence of structural and conformation change which prevents the accessibility and recognition of the cognate antibody and abrogation of signals. On the other hand, the biotin-biding peptide (Biot-tag) and the Strep II tag affinities did not substantially change when presented at both positions of other peptides (N- or C-terminus). In preferred embodiments, the Strep II tag and Biot-tag are utilized as transducer peptides because they provide flexibility in positioning. FIG. 14 provides a schematic representation of the general organization of the recombinant Qβ phage with the probe insert chimeric epitope insert and Step II, Biot, and LPETG tags.

### Example 4: Biosensor to Detect SARS Coronaviruses

Knowing the crucial role played by the highly glycosylated spike (S) protein of both coronaviruses (SARS-COV and SARS-COV-2), the causative agent of human severe acute respiratory syndrome (SARS) disease in mediating infection, we engineered RNA Qβ phage to map and elucidate the S protein domains recognizing antibody.

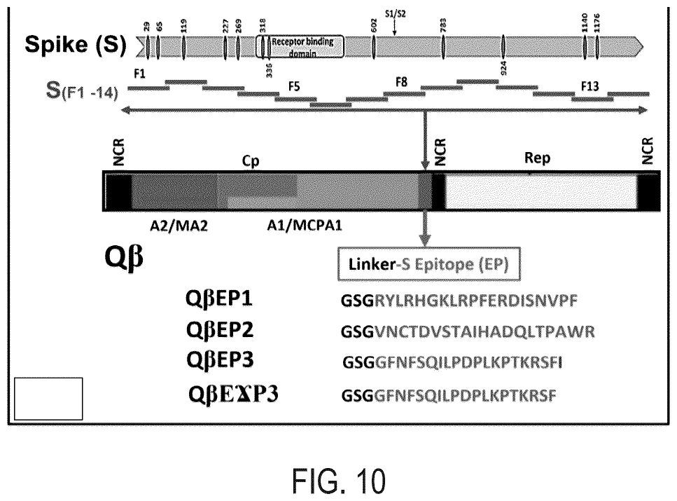

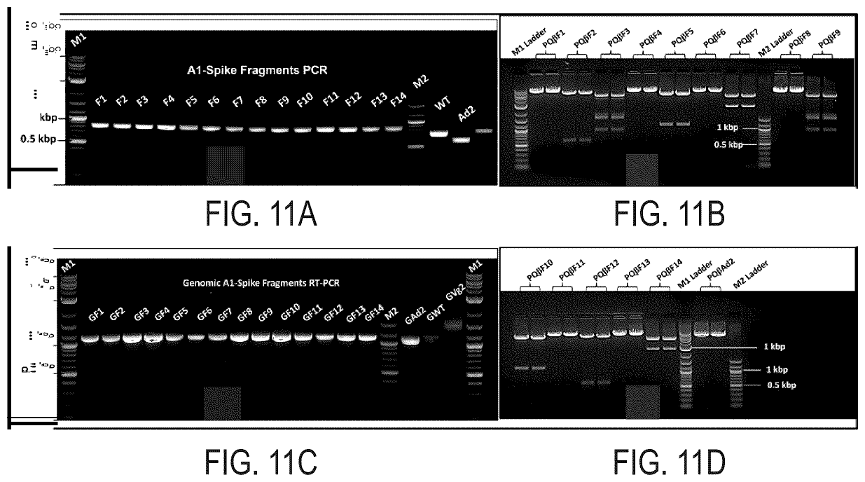

The C-terminus of the A1 protein was engineered to display a library of 14 overlapping peptide fragments derived from the spike (S) protein of SARS-COV (FIG. 10). Sequential fragments of S protein were stably inserted into the Qβ cDNA. During this process, restriction sequences of enzymes including Bpu10I and NsiI, were productively built into both the insert (each S fragment) and the plasmid vector (pQβ8). Likewise, plasmid vector expression cassettes were generated bearing an A1 gene modified in its C-terminus to display a library fragment of the S protein. As shown in FIG. 11A, through sequential modification of the A1 genome, we successfully increased from 150 bp to 300 bp the length of the DNA gene fused with its C-terminus. The modified C-terminus of the 500 bp A1 genome was effectively fused with 300 bp and tolerance of the Qβ genome for such long inserted DNA (300 bp) was established. The overall impact of this process was the permanent modification of the A1 gene from 500 bp to 800 bp within the recombinant phage display vector generated for each fragment. Finally, a restriction site was built in between the A1 natural stop codons (TAG and TAA) and the NsiI cloning enzyme site prior to clones/plasmids analysis. A total of fourteen fragments of S protein were effectively fused to A1 separately and built into the phage cDNA. In FIGS. 11B and 11D, a restriction enzyme gel analysis is shown for each fragment with the expected length. The recombinant plasmid library containing the various S fragments was sequenced, analyzed, and shown to contain the desired designed frame and relevant features necessary for the expression of recombinant phages.

To produce the first generation of recombinant Qβ phage, all plasmid vectors and constructed variants were transformed into E. coli DH5α or HB101, which are F− bacteria lacking the pilus appendage for reinfection. Additionally, using F− bacteria with plasmids with expression cassettes under the T7 promotor ensures exclusive usage of a high-fidelity DNA replication system that leads to plasmid transcription, resulting in a phage genome without premature evolutionary events within the phage expression system. Recombinant phage titers varying between 10³-10⁵ pfu/ml was obtained for various fragments, respectively (Table 3). The fragments 13 and 14 (F13 and F14) were poorly expressed and generated the lowest phage titer. The genome of each variant was analyzed by RT-PCR, agarose gel electrophoresis, and sequencing reactions. The results showed each variant containing the expected appropriate DNA fragment size (FIG. 11C) when compared to the wild type and the control phage with a deleted A1. A fragment size of 1500 bp was formed consisting of the A1, S fragment, inter-regional section upstream of the replicase, and partial replicase genes, respectively. To confirm the presence of the corresponding S fragment gene within the plaques obtained each phage variant's cDNA was sequenced. The results show that recombinant phages from each plaques corresponded specifically with their respective genes (genotype) fused in frame with the A1.

Recombinant human ACE2 (rhACE2), the natural receptor of SARS-COV, and anti-S antibodies were immobilized as target prior to the selective biopanning procedure. All recombinant phages presenting various S fragments, respectively, were incubated either with rhACE2 or anti-S antibody (Table 3).

TABLE 3
Comparison of reactivity of fragments of the SARS-CoV spike protein

| | | | | Opti- | | | |
|---|---|---|---|---|---|---|---|
| Fragment | Position | Known as | Platform | mization | Plasmids | Phages | Titer |
| F1 | S₁₋₁₀₀ | SP/NTD | — | — | pQβF1 | QβF1 | 10⁴ |
| F2 | S₈₅₋₁₈₅ | NTD | — | — | pQβF2 | QβF2 | 10⁵ |
| F3 | S₁₇₀₋₂₇₀ | NTD | — | — | pQβF3 | QβF3 | 10⁵ |
| F4 | S₂₅₅₋₃₅₅ | NTD-RBD | No Binding | — | pQβF4 | QβF4 | 10⁵ |
| F5 | S₃₄₀₋₄₄₀ | RBD/RBM | No Binding | — | PQβF5 | QβF5 | 10⁴ |
| F6 | S₄₂₅₋₅₂₅ | RBM/RBD | Bind to rhACE2 | 437-492 | pQβF6 | QβF6 | 10⁵ |
| F6 | S₄₂₅₋₅₂₅ | RBM/RBD | IgG | 441-460 | pQβF6 | QβF6 | 10⁵ |
| F7 | S₅₁₀₋₆₁₀ | S1 | IgG | 590-610 | pQβF7 | QβF7 | 10⁵ |
| F8 | S₅₉₅₋₆₉₅ | S1 | IgG | 604-635 | pQβF8 | QβF8 | 10⁵ |
| F9 | S₆₈₀₋₇₈₀ | S1/FP | — | — | pQβF9 | QβF9 | 10⁵ |
| F10 | S₇₆₅₋₈₆₅ | FP/S2 | IgG | 785-800 | pQβF10 | QβF10 | 10⁴ |
| F11 | S₈₅₀₋₉₅₀ | S2/HR1 | — | — | pQβF11 | QβF11 | 10⁵ |
| F12 | S₉₃₅₋₁₀₃₅ | HR1/S2 | — | — | pQβF12 | QβF12 | 10⁵ |
| F13 | S₁₀₂₀₋₁₁₄₀ | S2 | — | — | pQβF13 | QβF13 | 10³ |
| F14 | S₁₁₃₅₋₁₂₅₅ | HRW/TM/CP | — | — | pQβF14 | QβF14 | 10³ |

After several washes to eliminate nonspecific binders, target bound recombinant phages (upon hACE2 or IgG) were amplified using a log phase culture of either E. coli Q13 or K12. Elution was achieved therefore by recombinant phage infection and the genomic characterization was followed by sequencing. Through this process the recombinant phages harboring an S fragment 6 (GF6) were identified which bind specifically to rhACE2. Other recombinant phages containing S fragments including 6, 7, 8, and 10 (GF6, GF7, GF8 and GF10), respectively were found to bind specifically to anti-S antibodies. The GF6 affinity to the anti-S antibodies was comparatively low. requiring more recombinant phages initially for selection. The selected recombinant phages bearing target specific S fragments were confirmed quantitatively by ELISA. The differential binding activity of recombinant phages to the anti-S antibodies and the rhACE2 indicated that the recombinant phages exposing the corresponding functional peptide sequences (phenotype) defined by the inserted gene in the phage genome.

To determine the motif within S recognizing the rhACE2, the GF6 recombinant phages bearing the S fragment 6 were subjected to sequential deletion yielding mutants with 5, 3, and 2 residues from the N- and then C-termini. The mutant's genes were synthesized by PCR and cloned into our optimized expression cassette. The recombinant plasmid was introduced into E. coli HB101 for expression and phage production. Each of the newly generated mutant recombinant phages were used for affinity analysis with rhACE2 as an agonist. A resultant motif with residues between 437-492 AA (S437-492) within GF6 (of S fragment 6) was identified as the smallest rhACE2-recognizing motif that retained the full selective binding activity using a quantitative ELISA. Any other deletion mutants bound weakly or almost lost the binding activity to rhACE2, indicating that the amino acids between positions 437-492 AA are essential in target host receptor recognition. The GF6 motif (S₄₃₇₋₄₉₂) was subjected to panning using the immobilized rhACE2. Following elution by infection as previously described, recombinant phages, with a titer of 10₅ pfu/ml was obtained with a round of amplification using E. coli Q13. The resultant recombinant phage was further subjected to quantitative ELISA using a two-fold serial dilution of rhACE2. The results showed a corresponding increase in absorbance (OD) of HRP conjugated anti-hACE2 antibodies with increasing concentration of rhACE2. A plateau was observed at a concentration of rhACE2 between 1.25 and 2.50 μg/ml. The absorption curve was characteristic of the affinity between the SARS-COV S receptor binding domain and the hACE2. Thus, our finding demonstrates the expected hACE2 binding motif within the S protein.

To determine the selective anti-S antibody reactive epitopes for the recombinant phages GF6, GF7, GF8, and GF10, a similar series of sequential N- or C-terminal deletion mutants of each fragment of the S region gene were generated by PCR and fused in frame with the A1 to reconstruct the corresponding recombinant plasmids, respectively. All these deletion mutants were performed sequentially from 10, 5, 3, and 2 residues from each end of the fragment and the affinity of the newly obtained recombinant phages to anti-S antibodies were analyzed, respectively. As a result, fragment 6 situated between residues 441-460 AA at position (S₄₄₁₋₄₆₀) were found to retain a correspondingly increasing affinity for the anti-S antibodies. By deleting the C-terminus of F7 and the N-terminus of F8, the affinity to anti-S antibody was abolished, notably in quantitative ELISA, suggesting an overlapping region between both fragments. As for fragments 7 and 8 residues situated between 601-620 AA (S₆₀₁₋₆₂₀) retained optimal affinity for anti-S antibodies while in fragment 10, it was instead residues 781-800 AA (S781-800). Sequentially, the residues starting from fragments 6, 7-8, and 10were named epitope 1 (EP1), epitope 2 (EP2), and epitope 3 (EP3), respectively. For all the epitopes obtained, deletion mutants of a single amino acid in the C-terminal dramatically weaken or almost completely abrogate the binding activity to the anti-S antibodies in contrast to similar deletions in the N terminal region. An example for EP3 is presented in FIG. 10, where a simple deletion mutant of isoleucine residue reduces the OD in ELISA by half. This indicates that the epitope is located at residues situated in the carboxyl terminal of EP1, EP2, and EP3, respectively, while the amino terminal is an extension of the platform used. The results of the fine epitope mapping are presented and summarized in Table 4.

TABLE 4
Comparison of residues position, sequence,
and rank in affinity to anti-S
antibodies of the different epitopes
mapped of the SARS-CoV spike protein.

| | | Segment | |
|---|---|---|---|
| Residue | Amino acid | on the S | Rank in |
| position | sequence | protein | affinity |
| S₄₄₁₋₄₆₀ | RYLRHGKLRP | S1 | 1st |
| | FERDISNVPF | | |
| | (SEQ ID NO: 20) | | |
| S₆₀₁₋₆₂₀ | VNCTDVSTAI | S1 | 2nd |
| | HADQLTPAWR | | |
| | (SEQ ID NO: 21) | | |
| S₇₈₁₋₈₀₀ | GFNFSQILPD | S2 | 3rd |
| | PLKPTKRSFI | | |
| | (SEQ ID NO: 22) | | |

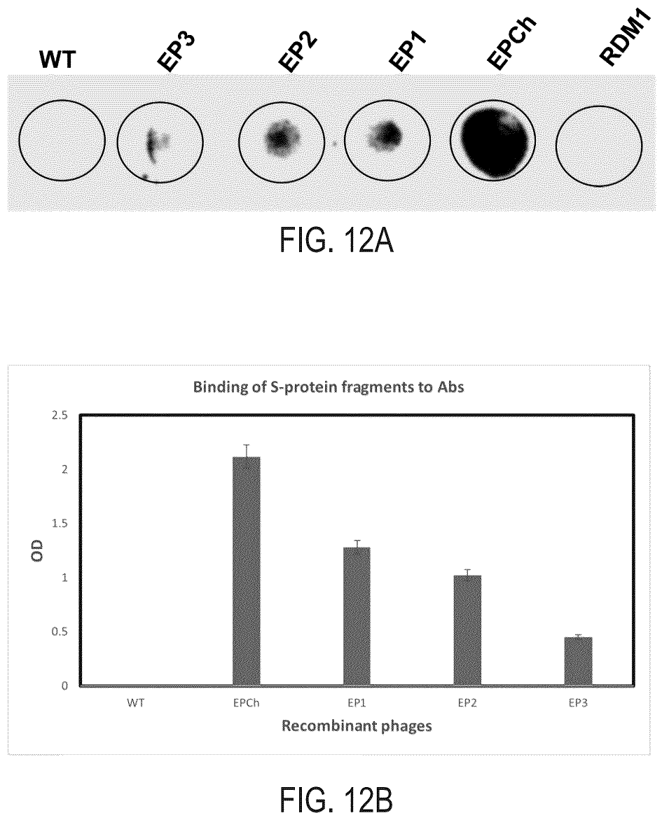

The recombinant phages bearing mapped epitopes specific to anti-S antibody from the identified fragments of SARS-COV S protein were subjected to further analysis using anti-S protein specific antibodies in ELISA and dot blotting. A three-dimensional (3D) computer simulation was used to analyze the fusion and exposition of the epitopes on A1. The 3D structural modeling indicated that each epitope is displayed without major impact on the A1 structure on the C-terminal end of the A1 minor coat protein as shown. The results show that all three phages bearing epitope motifs (EP1, EP2, and EP3) interacted with the antibody with different binding affinity (FIG. 12A). Their reactivity to the cognate antibody and the portion of the spike protein involved are depicted in Table 4, in comparison with other fragments and the wild type. By dot blotting visualization and quantitative ELISA analysis, EP1 showed the highest affinity to anti-S antibodies followed by EP2 and EP3, respectively. The results from dot blotting analysis were coincidental with those of ELISA (FIG. 12B), indicating that the chimeric epitope recognized with a higher affinity the anti-S antibodies than the mapped single epitopes. This order of affinity for anti-S antibodies was substantially different among the corresponding fragments (F6, F7/8, and F10). This result demonstrates that a major anti-S specific epitope (EP1 of F6) is found in the S protein buried within the receptor binding domain and is successfully exposed using our recombinant phage platform. The result was in conformity with the 3D structural simulation of the recombinant A1.

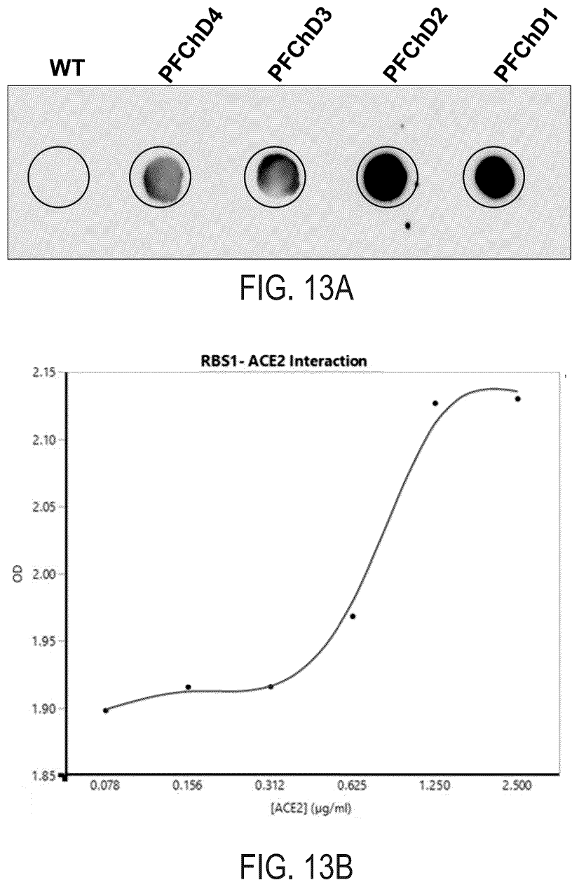

A chimeric construct of all three epitopes (EPCh) was generated in sequential order, joined by linkers, and analyzed. EPCh which is the combination of the three other epitopes (EP1, EP2, EP3) described above showed strong binding activity to the antibody, followed by EP1, EP2, and EP3 in dot blotting analysis (FIG. 13A). The same effect was observed in quantitative ELISA (FIG. 13B), where the chimeric epitope (EPCh) showed the highest absorbance compared to the individual epitopes (EP1, EP2, EP3) as displayed on the surface of the phages. The binding activity of the chimeric epitope to the anti-S antibodies increased with the titer of the recombinant phage in dot blotting. This result further confirms the reactivities of the epitopes mapped and also showed that a chimeric (all in one) may duplicate at least the binding reactivity of any of the single epitopes (EP1, EP2, or EP3) displayed upon recombinant phages. Moreso, the recombinant phages displayed epitopes showed no effect on the viability, structure, morphology, and stability when compared to the wild type. The recombinant phages may be utilized in combination with a Biot-tag transducer as the complete biosensor.

### Equivalents and Scope

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references is hereby incorporated by reference, particularly for the teaching referenced herein.

Sequence Table

| SEQ | | |
|---|---|---|
| ID NO. | Sequence | Description |
| 1 | QQEKNEKDLLALDSWANLWNWFDITNWLWYIKIFIMIVGGLI | HIV probe |
| | CLRIIFAV | |
| 2 | SARGDLA | FMDV |
| | | probe |
| 3 | TNASSEVAVLYQDVNCTDVSTAIHADQLTPAWRIYSTGNN | SARS-CoV |
| | | probe |
| 4 | TNTSNQVAVLYQDVNCTEVPVAIHADQLTPTWRVYSTGS | SARS-CoV- |
| | | 2 probe |
| 5 | GGSGGSGGSGGS | Linker |
| 6 | GGSGG | Linker |
| 7 | HGHGWQIPVWPWGQG | biotin- |
| | | specific |
| | | binding |
| | | peptide |
| 8 | ttaagtcgataaatgcc(NNN)15agtaactaaggatgaaatgca | ABW1 |
| 9 | cagctattacgg | ABW2 |
| 10 | atcattgattcctacttt | ABW3 |
| 11 | aatgtccaattcaagctgtgatagtcgttcctcgtgctgaattcgtc | Au1 |
| | agtggttcctctcccgacagttagtaactaaggatgaaatgcatggg | |
| 12 | gtccaattcaagctgtgatagtcgttcctcgtaagcttacaggtacttca | Au2 |
| | gtcctcattgcaactccatacgtttagtaactaaggatgaaatgcatggg | |
| 13 | gtccaattcaagctgtgatagtcgttcctcgtaagcttatgagccctcac | Silica |
| | cctcatccgcgacaccatcacacctagtaactaaggatgaaatgcatggg | |
| 14 | aatgtccaattcaagctgtgatagtcgttcctcgtgctagcctgaccccg | Cds |
| | ctgaccaccagccatctgcgcagctagtaactaaggatgaaatgcatggg | |
| 15 | tgtccaattcaagctgtgatagtcgttcctcgtgctgtgattagcaacca | ZnS |
| | tggggcagcagccgccgcctgtagtaactaagcttgatgaaatgcatgt | |
| 16 | gtccaattcaagctgtgatagtcgttcctcgtgctggtcatcaccatc | 6xhis-tag |
| | atcatcacgggtcctagtaagctagcctaaggatgaaatgcatgtgg | |
| 17 | gtccaattcaagctgtgatagtcgttcctcgtgcagcggccatcatcat | Biotin-tag |
| | catcatcatggcagctagtaagctagcctaaggatgaaatgcatgtgg | |
| 18 | gtccaattcaagctgtgatagtcgttcctcgtgcgatgtggaatggctggat | Strep II-tag |
| | gaacgcgtgccgctggtggaaacctagtaactaagcttgatgaaatgcatgt | |
| 19 | aaatgtccaattcaagctgtgatagtcgttcctcgtgctgcta | Co |
| | gcgaagaagaagaatagtaactaaggatgaaatgcatgtctaa | |
| 20 | RYLRHGKLRPFERDISNVPF | spike |
| | | protein S1 |
| | | residues |
| | | 441-460 |
| 21 | VNCTDVSTAIHADQLTPAWR | spike |
| | | protein S1 |
| | | residues |
| | | 601-620 |
| 22 | GFNFSQILPDPLKPTKRSFI | spike |
| | | protein S1 |
| | | residues |
| | | 781-800 |
| 23 | GGSGGSGGSGGSHGHGWQIPVWPWGQG | QβA1-Biot |
| 24 | GGSGGSGGSGGSHGHGWQIPVWPWGQGGGSGGSARGDLA | QβA1-Biot- |
| | | FMDV |
| 25 | GGSGGSGGSGGSHGHGWQIPVWPWGQGGGSGGSARGDLAG | QβA1-Biot- |
| | SHHHHHH | FMDV-6H |
| 26 | GGSGGSGGSGGSDVEWLDERVPLVET | QβA1-Strep |
| 27 | GGSGGSGGSGGSDVEWLDERVPLVETGGSGGSARGDLA | QβA1- |
| | | Strep- |
| | | FMDV |
| 28 | GSGRYLRHGKLRPFERDISNVPF | QβEP1 |
| 29 | GSGVNCTDVSTAIHADQLTPAWR | QβEP2 |
| 30 | GSGGFNFSQILPDPLKPTKRSFI | QβEP3 |
| 31 | GSGGFNFSQILPDPLKPTKRSF | QβEXP3 |

I claim:

1. An RNA-colliphage Qβ biosensor for detection of an agent comprising
a probe, a transducer and an analyte,
wherein the probe is amino acid sequence unique to the agent; and
wherein the transducer is a detectable molecule;
wherein the analyte is a molecule that binds to the probe;
wherein the transducer is positioned at the N-terminus or C-terminus of the probe; and
further wherein the probe and transducer are expressed on the surface of the RNA-colliphage Qβ at the position of the RNA-colliphage Qβ A₁ protein.

2. The RNA-colliphage Qβ biosensor of claim 1 wherein the agent is a biological agent.

3. The RNA-colliphage Qβ biosensor of claim 2 wherein the biological agent is a virus.

4. The RNA-colliphage Qβ biosensor of claim 3 wherein the virus is HIV, FMDV, SARS-CoV, SARS-COV-2, EBOV, or LASV.

5. The RNA-colliphage Qβ biosensor of claim 1 wherein the probe is a sequence for an epitope of a surface protein of the agent.

6. The RNA-colliphage Qβ biosensor of claim 1 wherein the transducer is a peptide.

7. The RNA-colliphage Qβ biosensor of claim 6 wherein the peptide is selected from the group consisting of furan, Strep II tag, Sortase A, Cytein-maleine, and Biot tag.

8. The RNA-colliphage Qβ biosensor of claim 1 wherein the analyte is an IgG specific to the probe.

9. A method of detecting SARS-COV using the biosensor of claim 1, comprising the steps of exposing the biosensor to a sample containing an analyte specific to the agent for detection to;
allowing the analyte to bind to the biosensor probe;
detecting the presence of biosensor transducer;
comparing the detection of biosensor transducer present after exposure to the sample with a control detection of biosensor before exposure to the sample.

10. The method of claim 9 wherein the RNA-colliphage Qβ biosensor comprises
the probe SEQ ID NO:3 the transducer is Sortase A, and the transducer is positioned at the N-terminal of the probe; and
wherein the analyte is an anti-S antibody.

11. The method of claim 9 wherein the RNA-colliphage Qβ biosensor comprises
the probe is SEQ ID NO:3 the transducer is cysteine-maleine, and the transducer is positioned at the N-terminal of the probe; and
wherein the analyte is an anti-S antibody.
